(19) United States
(12) Patent Application Publication
(10) Pub. No.: US 2007/0141689 A1
(43) Pub. Date: Jun. 21, 2007

(54) Liquefaction process

(75) Inventors: Swapnil Bhargava, Raleigh, NC (US); Henrik Bisgard-Frantzen, Bagsvaerd (DK); Henrik Frisner, Vaerlose (DK); Anders Vikso-Nielsen, Slangerup (DK); Malcolm Johal, Raleigh, NC (US)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North American, Inc., Franklinton, NC (US)

(21) Appl. No.: 10/593,164

(22) PCT Filed: Mar. 18, 2005
(86) PCT No.: PCT/US05/09218

§ 371 Date: Oct. 18, 2006

Related U.S. Application Data
(60) Provisional application No. 60/554,615, filed on Mar. 19, 2004.
(60) Provisional application No. 60/575,133, filed on May 28, 2004.

(51) Int. Cl. C12P 7/06; C12N 9/32

(52) U.S. Cl. 435/161; 435/204

(57) ABSTRACT

The present invention relates to method of liquefying starch-containing material, wherein the method comprises the steps of (a) treating the starch-containing material with a bacterial alpha-amylase at a temperature around 70-90° C. for 15-90 minutes, and (b) treating the material obtained in step (a) with an alpha-amylase at a temperature between 60-80° C. for 30-90 minutes. The invention also relates to a process of producing a fermentation product, preferably ethanol, comprising a liquefaction step carried out according to the liquefaction method of the invention.

### CROSS-REFERENCE TO RELATED APPLICATIONS

This application claims priority from U.S. provisional application Ser. Nos. 60/575,133, filed on May 28, 2004, and 60/554,615, filed on Mar. 19, 2004, which are hereby incorporated by reference. This application contains a sequence listing, which is hereby incorporated by reference.

### FIELD OF THE INVENTION

The present invention relates to an improved method of liquefying starch-containing material suitable as step in processes for producing syrups and fermentation products, such as especially ethanol. The invention also relates to processes for producing a desired fermentation product, preferably ethanol, comprising liquefying starch-containing starting material in accordance with the liquefaction method of the invention.

### BACKGROUND OF THE INVENTION

Liquefaction is a well known process step in the art of producing syrups and fermentation products, such as ethanol, from starch-containing materials. During liquefaction starch is converted to shorter chains and less viscous dextrins. Generally liquefaction involves gelatinization of starch simultaneously with or followed by addition of alpha-amylase.

WO 02/38787 (Novozymes) disclose a method of producing ethanol by fermentation comprising carrying out secondary liquefaction in the presence of a thermostable acid alpha-amylase or a thermostable maltogenic acid alpha-amylase.

Even though liquefaction has already been improved significantly there is still a need for improving liquefaction suitable for syrup and fermentation product producing processes.

### SUMMARY OF THE INVENTION

The object of the present invention is to provide an improved method of liquefying starch-containing material suitable as a step in processes for producing syrups and fermentation products, such as especially ethanol. The invention also provides a process for producing a desired fermentation product which includes a liquefaction method of the invention.

The present inventors have found that when liquefaction is carried out on starch-containing material in accordance with the present invention a number of advantages are obtained. For instance, the inventors have shown that a DE above 20 may be obtained without using more enzyme than corresponding prior art processes which reaches a DE around 12. Further, reduced viscosity was observed. This eases handling of the liquefied material and reduces the cost of pumping the liquefied material to down stream process equipment such as a fermentor. Furthermore, the enzyme cost is also reduced. It was also found that the sugar profile of the liquefied mash had a decreased DP₄₊ content and increased DP₁₋₃ content compared to corresponding prior art methods using higher amounts of enzyme. The higher DP₁₋₃ content makes the liquefied mash easier and potentially faster to ferment by a fermenting organism, such as yeast, during, e.g., ethanol fermentation. This could be attributed to the fact that small sugars are released pertaining to the (acid) alpha-amylase action. These small sugars, e.g., glucose, maltose and maltotriose, can be directly metabolized by the fermenting organism and therefore makes SSF more effective and fast.

Further, also the residual DP₄₊ content were after fermentation found to be higher than in corresponding prior art processes. This indicates a better utilization of the starch-containing starting material.

The abbreviation “DE” stands for “Dextrose Equivalent” and is a measure for reducing ends on C₆ carbohydrates. Pure dextrose (glucose) has a DE of 100. Dextrose is a reducing sugar. Whenever an amylase hydrolyzes a glucose-glucose bond in starch, two new glucose end-groups are exposed. At least one of these can act as a reducing sugar. Therefore the degree of hydrolysis can be measured as an increase in reducing sugars. The value obtained is compared to a standard curve based on pure glucose—hence the term dextrose equivalent. In other words: DE (dextrose equivalent) is defined as the amount of reducing carbohydrate (measured as dextrose-equivalents) in a sample expressed as w/w % of the total amount of dissolved dry matter.

According to the first aspect the invention relates to a method of liquefying starch-containing material, wherein the method comprises the steps of:
(a) treating starch-containing material with a bacterial alpha-amylase at a temperature around 70-90° C. for 15-90 minutes,
(b) treating the material obtained in step (a) with an alpha-amylase at a temperature between 60-80° C. for 30-90 minutes.

The term “mash” is used for liquefied starch-containing material, such as liquefied whole grain.

In one embodiment of the invention the starch-containing material is jet-cooking at 90-120° C., preferably around 105° C., for 1-15 minutes, preferably for 3-10 minute, especially around 5 minutes, before step (a).

After step (b) the mash has a DE value above 16, preferably above 18, especially above 20, such as a DE value in the range from 16 to 30, preferably in the range from 18 to 25.

In a second aspect the invention provides a process of producing a fermentation product, especially ethanol, from starch-containing material by fermentation, said process comprises the steps of:
(i) liquefying starch-containing material according to the liquefaction method of the invention;
(ii) saccharifying the liquefied mash obtained;
(iii) fermenting.

Optionally the ethanol is recovery after fermentation. In an embodiment the saccharification and fermentation is carried out as a simultaneous saccharification and fermentation process (SSF process).

### BRIEF DESCRIPTION OF THE INVENTION

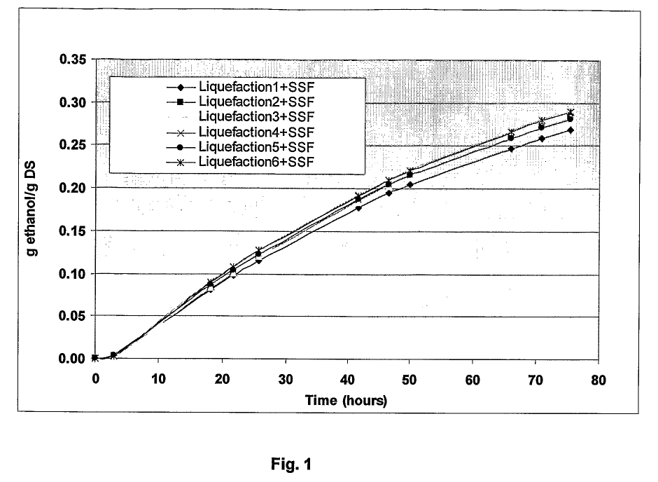

FIG. 1: Ethanol yields from six liquefaction treatments with 0.3 AGU/g DS of Glucoamylase SF.

### DESCRIPTION OF THE INVENTION

The present invention provides an improved liquefaction method suitable as a step in processes for producing fermentation products such as especially ethanol. The invention also relates to a process of producing a fermentation product, especially ethanol, comprising a liquefaction method of the invention. Where the end product is ethanol it may be used as, e.g., fuel ethanol; drinking ethanol, i.e., potable neutral spirits; or industrial ethanol.

Liquefaction

According to the present invention “liquefaction” is a process step in which starch-containing material, preferably milled (whole) grain, is broken down (hydrolyzed) into maltodextrins (dextrins).

Initially an aqueous slurry containing preferably from 10-40 wt-%, especially 25-35 wt-% starch-containing material is prepared. The starch-containing material is preferably milled whole grain. Then the starch-containing material is incubated with a bacterial alpha-amylase, preferably one or more Bacillus alpha-amylases, and may in one embodiment be followed by a jet-cooking step carried out between 90-120° C., preferably around 105° C., for 1 - 15 minutes, preferably for 3-10 minutes, especially around 5 minutes, to complete gelatinization of the slurry. However, it is to be understood that the method of the invention may also be carried out without a jet-cooking step. After incubation with bacterial alpha-amylase, with or without jet-cooking, the temperature is adjusted to 60-80° C. and the material is incubated for 30 to 90 minutes in the presence of an alpha-amylase, preferably an acid alpha-amylase, especially a fungal acid alpha-amylase, to finalize hydrolysis (secondary liquefaction).

Consequently, in the first aspect the invention provides a method for liquefying starch-containing material comprising the steps of:
(a) treating starch-containing material with a bacterial alpha-amylase at a temperature around 70-90° C. for 15-90 minutes,
(b) treating the material obtained in step (a) with an alpha-amylase at a temperature between 60-80° C. for 30-90 minutes.

A liquefaction method of the invention is typically carried out at pH 4.5-6.5, in particular at a pH between 5 and 6.

The alpha-amylase may be any alpha-amylase, preferred an alpha-amylase mentioned in the section “Alpha-amylases” below.

Starch-Containing Material

The starch-containing material used according to the present invention may be selected from the group consisting of: tubers, roots and whole grain, and any combinations of the forgoing. In an embodiment, the starch-containing material is obtained from cereals. The starch-containing material may, e.g., be selected from the groups consisting of corns, cobs, wheat, barley, cassava, sorghum, rye, milo and potatoes; or any combination of the forgoing.

If the liquefaction method of the invention is included in an ethanol process of the invention, the raw starch-containing material is preferably whole grain or at least mainly whole grain. A wide variety of starch-containing whole grain crops may be used as raw material including: corn (maize), milo, potato, cassava, sorghum, wheat, and barley. Thus, in one embodiment, the starch-containing material is whole grain selected from the group consisting of corn (maize), milo, potato, cassava, sorghum, wheat, and barley; or any combinations thereof. In a preferred embodiment, the starch-containing material is whole grain selected from the group consisting of corn, wheat and barley or any combinations thereof.

The raw material may also consist of or comprise a side-stream from starch processing, e.g., C₆ carbohydrate containing process streams that are not suited for production of syrups.

Milling

In a preferred embodiment of the invention the starch-containing material is milled before step (a), i.e., before the primary liquefaction. Thus, in a particular embodiment, the liquefaction method further comprises—prior to the primary liquefaction step (i.e., prior to step (a),—the steps of:
i. milling of the starch-containing material, such as whole grain;
ii. forming a slurry comprising the milled starch-containing material and water.

The starch-containing material, such as whole grain, is milled in order to open up the structure and allowing for further processing. Two processes of milling are normally used in ethanol production processes: wet and dry milling. The term “dry milling” denotes milling of the whole grain. In dry milling the whole kernel is milled and used in the remaining part of the process. Wet milling gives a good separation of germ and meal (starch granules and protein) and is with a few exceptions applied at locations where there is a parallel production of syrups. Dry milling is preferred in processes aiming at producing ethanol. The term “grinding” is also understood as milling. In a preferred embodiment of the invention dry milling is used. However, it is to be understood that other methods of reducing the particle size of the starch-containing material are also contemplated and covered by the scope of the invention.

Process for Producing a Fermentation Product

A process of the invention generally involves the steps of liquefaction, saccharification, fermentation and optionally recovering the fermentation product, such as ethanol, preferably by distillation.

According to this aspect, the invention relates to a process of producing a fermentation product, preferably ethanol, from starch-containing material by fermentation, said method comprises the steps of:
(i) liquefying said starch-containing material according to the liquefaction method of the invention;
(ii) saccharifying the liquefied mash obtained in step (i)
(iii) fermenting.

In an embodiment the saccharification and fermentation steps ii) and iii) are carried out as a simultaneous saccharification and fermentation process (SSF process). In a preferred embodiment of the invention starch-containing raw material, such as whole grain, preferably corn, is dry milled in order to open up the structure and allow for further processing. The mash has before step (ii), i.e., after step (i), with or without jet-cooking before step i), a DE value of above 16, preferably above 18, especially above 20, such as a DE value in the range from 16 to 30, preferably in the range from 18 to 25.

A specific embodiment of the process of the invention comprises the steps of;
1) liquefying starch-containing material in accordance with the liquefaction method of the invention;
2) liquefying the material obtained in step 1) in the presence of an alpha-amylase having an amino acid sequence which has at least 70% identity to SEQ ID NO:1; and
3) saccharifying the material obtained; and
4) fermenting to produce a fermentation product, preferably ethanol; wherein the steps 1), 2), 3) and 4) is performed in the order 1), 2), 3), 4) or wherein 4) is performed simultaneously with or following 3).

In a preferred embodiment a jet-cooking step, as defined above, is included before step 1). In a preferred embodiment the alpha-amylase used in step ii) is at least 75%, 80%, 85% or at least 90%, e.g., at least 95%, at least 97%, at least 98%, or at least 99% identity to SEQ ID NO:1.

The mash has after step 2), with or without jet-cooking before step 1), a DE value of above 16, preferably above 18, especially above 20, such as a DE value in the range from 16 to 30, preferably in the range from 18 to 25.

Saccharification

“Saccharification” is a step in which the maltodextrin (such as, product from the liquefaction) is converted to low molecular sugars DP₁₋₃ (i.e., carbohydrate source) that can be metabolized by a fermenting organism, such as, yeast. Saccharification is well known in the art and is typically performed enzymatically using at least a glucoamylase or one or more carbohydrate-source generating enzymes as will be defined below. The saccharification step comprised in the process for producing ethanol of the invention may be a well known saccharification step in the art. In one embodiment glucoamylase, alpha-glucosidase and/or acid alpha-amylase is used for treating the liquefied starch-containing material. A full saccharification step may last up to from 20 to 100 hours, preferably about 24 to about 72 hours, and is often carried out at temperatures from about 30 to 65° C., and at a pH between 4 and 6, normally around pH 4.5-5.0. However, it is often more preferred to do a pre-saccharification step, lasting for about 40 to 90 minutes, at temperature of between 30-65° C., typically about 60° C., followed by complete saccharification during fermentation in a simultaneous saccharification and fermentation process (SSF). The most widely used process for ethanol production is the simultaneous saccharification and fermentation (SSF) process, in which there is no holding stage for the saccharification, meaning that fermenting organism, such as yeast, and enzyme(s) is(are) added together. In SSF processes, it is common to introduce a pre-saccharification step at a temperature between 40 and 60° C., preferably around 50° C., just prior to the fermentation.

Fermentation Product

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H2 and CO2); antibiotics (e.g., penicillin and tetracycline); en-zymes; vitamins (e.g., riboflavin, B12, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes, as are well known in the art. Preferred fermentation processes are anaerobic fermentation processes

Fermentation

In a process of the invention the fermenting organism is preferably yeast, which may be applied to the saccharified material.

The term “fermenting organism” refers to any organism suitable for use in a desired fermentation process. Suitable fermenting organisms are according to the invention capable to ferment, i.e., convert sugars, such as glucose or maltose, directly or indirectly into the desired fermentation product, preferably ethanol. Examples of fermenting organisms include fungal organisms, such as yeast. For ethanol production preferred yeast includes strains of Saccharomyces spp., and in particular Saccharomyces cerevisiae. Commercially available yeast includes, e.g., RED STAR®/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties). In preferred embodiments, yeast is applied to the saccharified mash. Fermentation is ongoing for 24-96 hours, such as typically 35-65 hours. In preferred embodiments, the temperature is generally between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

Recovery of Ethanol

Optionally the ethanol is recovery after fermentation, preferably by including the step of;

(iv) distillation to obtain the ethanol;
   wherein the fermentation in step (iii) and the distillation in step (iv) is carried out simultaneously or separately/sequential; optionally followed by one or more process steps for further refinement of the ethanol.
   Starch Conversion

The liquefaction method of the invention may also be included in a starch conversion process for producing syrup such as glucose, maltose, fructose syrups, e.g., high fructose syrup (HFS), malto-oligosaccharides and isomalto-oligosaccharides. Suitable starting materials are exemplified in the “Starch-containing material”-section above. The process comprises a liquefaction method of the invention followed by saccharification in order to, e.g., release sugar from the non-reducing ends of the starch or related oligo- and polysaccharide molecules in the presence of carbohydrate-source generating enzyme.

Consequently, this aspect of the invention relates to a process of producing syrup from starch-containing material, comprising
(a) liquefying starch-containing material in accordance with the liquefaction method of the invention,
(b) saccharifying the liquefied material.
   To produce, e.g., fructose an isomerization step is included. Optionally the syrup may be recovered from the saccharified material obtained in step (b) or after an additional step.

Details on suitable liquefaction and saccharification conditions can be found above.

Alpha-Amylases

According to the invention preferred any alpha-amylases may be used. Preferred alpha-amylases are of fungal or bacterial origin.

Bacterial Alpha-Amylase

The bacterial alpha-amylase may be any bacterial alpha-amylase.

In a preferred embodiment the Bacillus alpha-amylase is derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase (BLA) shown in SEQ ID NO: 4 in WO 99/19467, the Bacillus amyloliquefaciens alpha-amylase (BAN) shown in SEQ ID NO: 5 in WO 99/19467, and the Bacillus stearothermophilus alpha-amylase (BSG) shown in SEQ ID NO: 3 in WO 99/19467. In an embodiment of the invention the alpha-amylase is an enzyme having a degree of identity of at least 60%, preferably at least 70%, more preferred at least 80%, even more preferred at least 90%, such as at least 95%, at least 96%, at least 97%, at least 98% or at least 99% identity to any of the sequences shown as SEQ ID NOS: 1, 2, 3, 4, or 5, respectively, in WO 99/19467. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873 -see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted 1181* +G182* +N193F) compared to the wild-type BSG alpha-amylase amino acid sequence set forth in SEQ ID NO:3 disclosed in WO 99/19467.

A hybrid alpha-amylase specifically contemplated comprises 445 C-terminal amino acid residues of the Bacillus licheniformis alpha-amylase (shown as SEQ ID NO: 4 in WO 99/19467) and the 37 N-terminal amino acid residues of the alpha-amylase derived from Bacillus amyloliquefaciens (shown as SEQ ID NO: 3 in WO 99/194676), with one or more, especially all, of the following substitution:

G48A+T49l+G 107A+H 156Y+A181 T+N 190F+l201 F+A209V+Q264S (using the Bacillus licheniformis numbering). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467). The bacterial alpha-amylase may be added in an amount well-known in the art. When measured in KNU units the alpha-amylase activity is preferably present in an amount of 0.5-5,000 NU/g of DS, in an amount of 1-500 AAU/kg of DS, or more preferably in an amount of 5-1,000 KNU/kg of DS, such as 10-100 KNU/kg DS.

Fungal Alpha-Amylase

The fungal alpha-amylase may be any fungal alpha-amylase. Preferred fungal alpha-amylases include alpha-amylases derived from a strain of Aspergillus, such as, Aspergillus oryzae, Aspergillus niger, or A. kawashii alpha-amylases. In a preferred embodiment, the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. A commercially available acid fungal amylase is SP288 (available from Novozymes A/S, Denmark).

In an embodiment the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

A preferred acid fungal alpha-amylase is a Fungamyl-like alpha-amylase. In the present disclosure, the term “Fungamyl-like alpha-amylase” indicates an alpha-amylase which exhibits a high identity, i.e., more than 50%, 55%, 60%, 65%, 70%, 75%, 80%, 85% 90%, 95 or even 99% identical to the amino acid sequence shown in SEQ ID NO: 10 in WO 96/23874. When used as a maltose generating enzyme fungal alpha-amylases may be added in an amount of 0.001-1.0 AFAU/g DS, preferably from 0.002-0.5 AFAU/g DS, preferably 0.02-0.1 AFAU/g DS.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variants of set acid fungal amylase having at least 70% identity, such as at least 80% or even at least 90%, 95%, 96%, 97%, 98% or 99% identity thereto is contemplated. In an embodiment the acid fungal alpha-amylase is the one disclosed in SEQ ID NO: 1, or a sequence being at least 70% identical, preferably at least 75%, 80%, 85% or at least 90%, e.g. at least 95%, 97%, 98%, or at least 99% identity to SEQ ID NO:1.

Fungal acid alpha-amylase are preferably added in an amount of 0.001-10 AFAU/g of DS, in an amount of 0.01-0.25 AFAU/g of DS, or more preferably in an amount of 0.05-0.20 AFAU/kg of DS, such as around 0.1 AFAU/k DS.

Commercial Alpha-Amylases

Preferred commercial compositions comprising an alpha-amylase include MYCO-LASE™ from DSM; BAN™, TERMAMYL™ SC, FUNGAMYL™, LIQUOZYME™ X and SAN™ SUPER, SAN™ EXTRA L from Novozymes A/S, Denmark) and CLARASE™ L-40,000, DEX-LO™, SPEYME FRED, SPEZYME™ ETHYL, SPEZYME™ AA, and SPEZYME™ DELTA M (Genencor Int., USA), and the acid fungal alpha-amylase sold under the trade name SP 288 (available from Novozymes A/S, Denmark).

Carbohydrate-Source Generating Enzyme

The term “carbohydrate-source generating enzyme” includes glucoamylase (being a glucose generator), beta-amylase and maltogenic amylases (being maltose generators). A carbohydrate-source generating enzyme is capable of providing energy to the fermenting organism(s) used in a process of the invention for producing the desired fermentation product, especially ethanol. The generated carbohydrate may be converted directly or indirectly to the desired fermentation product. The carbohydrate-source generating enzyme may be mixtures of enzymes falling within the definition. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50.

Examples of contemplated glucoamylases, maltogenic amylases, and beta-amylases are set forth in the sections above and below.

Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381, WO 00/04136 add WO 01/04273 (from Novozymes, Denmark); the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Athelia rolfsii (previously denoted Corticium rolfsil) glucoamylase (see U.S. Pat. No. 4,727,026 and (Nagasaka,Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Patent No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, such as 2 AGU/g DS.

Beta-Amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S under the tradename MALTOGENASE™. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

Production of Enzymes

The enzymes referenced herein may be derived or obtained from any suitable origin, including, bacterial, fungal, yeast or mammalian origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g., by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used according to the present invention may be in any form suitable for use in the processes described herein, such as, e.g., in the form of a dry powder or granulate, a non-dusting granulate, a liquid, a stabilized liquid, or a protected enzyme. Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, lactic acid or another organic acid according to established process. Protected enzymes may be prepared according to the process disclosed in EP 238,216.

Even if not specifically mentioned in context of a method or process of the invention, it is to be understood that the enzyme(s) or agent(s) is(are) used in an “effective amount”.

Materials and Methods

Enzymes:

Bacterial Alpha-amylase A: Bacillus stearothermophilus alpha-amylase variant with the mutations: l181*+G182*+N193F disclosed in U.S. Pat. No. 6,187,576 and available on request from Novozymes A/S, Denmark.
Fungal acid alpha-amylase B: Aspergillus niger alpha-amylase disclosed in SEQ ID NO: 1 and available from Novozymes A/S.
Glucoamylase T: Glucoamylase derived from Talaromyces emersonii and disclosed as SEQ ID NO: 7 in WO 99/28448.
Glucoamylase SF: Balanced blend of Aspergillus niger glucoamylase and A. niger acid alpha-amylase having a ratio between AGU and AFAU of approx. 9:1.
   Stock Solution for Iodine Method:
0.1N I₂
dissolve 1.3 g I₂ and 2.0 g KI into 100 mL DI water
   Methods:
   Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C. +/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of color is therefore directly proportional to the concentration of starch. Amylase activity is determined using reverse colorimetry as a reduction in the concentration of starch under specified analytic conditions. 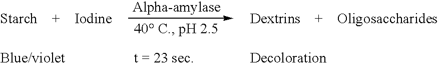
Standard conditions/reaction conditions: (per minute)
Substrate: Starch, approx. 0.17 g/L
Buffer: Citate, approx. 0.03 M
Iodine (I₂): 0.03 g/L
CaCI₂: 1.85 mM
pH: 2.50±0.05
Incubation temperature: 40° C.
Reaction time: 23 seconds
Wavelength: lambda=590 nm
Enzyme concentration: 0.025 AFAU/mL
Enzyme working range: 0.01-0.04 AFAU/mL

If further details are preferred these can be found in EB-SM-0259.02/01 available on request from Novozymes A/S, Denmark, and incorporated by reference.

Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.
Standard conditions/reaction conditions:
Substrate: Soluble starch. Concentration approx. 20 g DS/L.
Buffer: Citrate, approx. 0.13 M, pH=4.2
Iodine solution: 40.176 g potassium iodide +0.088 g iodine/L
City water 15°-20°dH (German degree hardness)
pH: 4.2
Incubation temperature: 30° C.
Reaction time: 11 minutes
Wavelength: 620 nm
Enzyme concentration: 0.13-0.19 AAU/mL
Enzyme working range: 0.13-0.19 AAU/mL

The starch should be Lintner starch, which is a thin-boiling starch used in the laboratory as calorimetric indicator. Lintner starch is obtained by dilute hydrochloric acid treatment of native starch so that it retains the ability to color blue with iodine. Further details can be found in EP0140410B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method described in the section 76-11 Starch-Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard conditions/reaction conditions:
Substrate: Soluble starch. Concentration approx. 16 g dry matter/L.
Buffer: Acetate, approx. 0.04 M, pH=4.3
pH: 4.3
Incubation temperature: 60° C.
Reaction time: 15 minutes
Termination of the reaction: NaOH to a concentration of approximately 0.2 g/L (pH˜9)
Enzyme concentration: 0.15-0.55 MU/mL

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

Standard Iodine Method

boil small aliquot (10-20 mLs) of liquefied material in a test tube for several minutes
cool in ice bath
add 10-12 drops of the iodine solution
mix and let sample sit in ice water for about 10 minutes
   Determination of Viscosity

The mash is heated to a temperature of 50-70° C., depending on the treatment. Following treatment viscosity is measured using a Haake VT02 rotation based viscosimeter. The unit of viscosity is centipois (cps), which is proportionally related to the viscosity level.

Determination of DE (Dextrose Equivalent)

The DE value is measured using Fehlings liquid by forming a copper complex with the starch using pure glucose as a reference, which subsequently is quantified through iodometric titration. DE (dextrose equivalent) is defined as the amount of reducing carbohydrate (measured as dextrose-equivalents) in a sample expressed as w/w % of the total amount of dissolved dry matter. It may also be measured by the neocuproine assay (Dygert, Li Floridana(1965) Anal. Biochem. No 368). The principle of the neocuproine assay is that CuSO₄ is added to the sample, Cu²⁺ is reduced by the reducing sugar and the formed neocuproine complex is measured at 450 nm.

Degree of Identity

The degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pair-wise alignment parameters were Ktuple=1, gap penalty=3, windows=5, and diagonals=5].

### EXAMPLES

### Example 1

Liquefaction with Bacterial and Acid Alpha Amylase

400 mL of ground corn slurry is liquefied with 50 NU/g dry solids (DS) Bacterial Alpha-Amylase A. The corn mash has about 30% dry substance (pH 5.4). The mash is heated to 85° C. and the viscosity and DE values are measured.

The mash is then treated with acid alpha-amylase B from Aspergillus niger having the amino acid sequence disclosed in SEQ ID NO:1. The enzyme loading is 0.10 AFAU/g dry solids. After 1.5 hours the viscosity and DE value are measured.

### Example 2

The Effect of Alpha-Amylase Addition During Liquefaction on SSF Performance:

To investigate the effect of alpha-amylase (in form of Acid Alpha-Amylase B) addition in liquefaction, six different conditions for liquefaction were tested. To begin with ground corn was used to make 30% slurry with tap water. The pH in all the liquefactions were adjusted to 5.4 using diluted H₂SO₄. In the first two liquefactions (controls), Bacterial Alpha-Amylase A (50 NU/g DS) was added and kept at 85° C. for 1.5 and 4.5 hours, respectively. In the second set of runs (Acid Alpha-Amylase B test), the same process was followed, but incubation time with Bacterial Alpha-Amylase A at 85° C. was reduced to 0.5 hours, the temperature was then lowered and Acid Alpha-Amylase B was added (0.050 and 0.10 AFAU/g DS). The mixture was then kept at 70° C. for 1 and 4 hours, respectively. Once the liquefaction was over, the reactions were stopped by adding 2 drops of HCI (4 N). Samples were withdrawn to analyze sugar profiles (using HPLC) and DE values. The liquefied samples were frozen and later subjected to SSF.

The effect of liquefaction treatment on SSF was evaluated via mini-scale fermentations. Samples after liquefaction were thawed and the pH was adjusted to 5.0 with diluted H₂SO₄. Approximately 4 grams of mash was added to 16 ml polystyrene tubes (Falcon 352025). Tubes were then dosed with the appropriate amount of Glucoamylase SF (0.3 AGU/g DS). Six replicates of each treatment were run. After dosing the tubes with enzyme, they were inoculated with 0.04 ml/g mash of yeast propagate that had been grown for 21 hours on corn mash. Vials were capped with a screw on lid which had been punctured with a very small needle to allow gas release and vortexed briefly before weighing and incubation at 32° C. Fermentation progress was followed by weighing the tubes over time. Tubes were vortexed briefly before each weighing. Weight loss values were converted to ethanol yield (g ethanol/g DS) (see FIG. 1) by the following formula:

$$g ethanol g DS = \frac{\begin{matrix}g {CO}_{2} weight loss \times \frac{1 mol {CO}_{2}}{44.0098 g {CO}_{2}} \times \\ \frac{1 mol ethanol}{1 mol {CO}_{2}} \times \frac{46.094 g ethanol}{1 mol ethatnol}\end{matrix}}{g corn in tube \times \% DSof corn }$$

Data from the liquefaction process shows that adding Acid Alpha-Amylase B in addition to Bacterial alpha-amylase A resulted in a significant increase in DE values.

| Treatment | enzyme1 | enzyme 2 | DE |
|---|---|---|---|
| 1 | BAA(50)-85° C.-1.5 hr | | 7.27 |
| 2 | BAA(50)-85° C.-4.5 hr | | 7.79 |
| 3 | BAA(50)-85° C.-0.5 hr | AAA(50)-70° C.-1 hr | 16.24 |
| 4 | BAA(50)-85° C.-0.5 hr | AAA(50)-70° C.-4 hr | 20.79 |
| 5 | BAA(50)-85° C.-0.5 hr | AAA(100)-70° C.-1 hr | 22.13 |
| 6 | BAA(50)-85° C.-0.5 hr | AAA(100)-70° C.-4 hr | 23.56 |

*BAA: Bacterial alpha-amylase
*AAA: Acid alpha-amylase

1. A method of liquefying starch-containing material, wherein the method comprises the steps of
(a) treating the starch-containing material with a bacterial alpha-amylase at a temperature around 70-90° C. for 15-90 minutes,
(b) treating the material obtained in step (a) with an alpha-amylase at a temperature between 60-80° C. for 30-90 minutes.

2. The method of claim 1, wherein the starch-containing material is jet-cooking at 90-120° C., preferably around 105° C., for 1-15 minutes, preferably for 3-10 minute, especially around 5 minutes, before step (a).

3-4. (canceled)

5. The method of claim 1, wherein the starch-containing material is selected from the group consisting of corn, cob, wheat, barley, rye, milo and potatoes; or any combination of these.

6-8. (canceled)

9. The method of claim 1, further comprising prior to step (a) the steps of;
i) milling of starch-containing material;
ii) forming a slurry comprising the milled material and water.

10. The method of any of the claim 9, wherein the milling step is a dry milling step.

11-12. (canceled)

13. The method of claim 1, wherein the bacterial alpha-amylase in step (a) is a Bacillus alpha-amylase.

14. The method of claim 1, wherein the alpha-amylase is step (b) is an acid alpha-amylase.

15. The method of claim 14, wherein the acid alpha-amylase is an alpha-amylase having an amino acid sequence which has at least 70% identity to SEQ ID NO:1.

16. (canceled)

17. The method of claim 1, wherein the mash obtained after step (b) has a DE value of above 16.

18. A process of producing ethanol from starch-containing material by fermentation, said process comprises:
(i) liquefying said starch-containing material as defined in claim 1;
(ii) saccharifying the liquefied mash obtained;
(iii) fermenting the material obtained in step (ii).

19. The process of claim 18, further comprising recovery of the ethanol.

20. The process of claim 18, wherein the saccharification and fermentation is carried out as a simultaneous saccharification and fermentation process (SSF process).

21-22. (canceled)

23. The process of claim 18, wherein the fermentation is carried out with a yeast.

24. The process of claim 18, wherein the fermentation is carried out in the presence of a carbohydrate-source generating enzyme.

25. The process of claim 24, wherein the carbohydrate-source generating enzyme is a glucoamylase.

26. The process of claim 18, said process comprising the steps of;
1) liquefying starch-containing material;
2) liquefying the material obtained in step 1) in the presence of an alpha-amylase having an amino acid sequence which has at least 70% identity to SEQ ID NO:1; and
3) saccharifying the material obtained in step 2); and
4) fermenting to produce ethanol; wherein the steps 1), 2), 3) and 4) is performed in the order 1), 2), 3), 4) or wherein 4) is performed simultaneously to or following 3).

27. The process of claims 26, wherein the mash obtained after step 2) has a DE value of above 16.

28. The method of claim 13, wherein the bacterial alpha-amylase is derived from a strain of Bacillus stearothermophilus.

29. The method of claim 14, wherein the acid alpha-amylase is an acid fungal alpha-amylase.

30. The method of claim 29, wherein the acid fungal alpha-amylase is derived from Aspergillus spp.

31. The method of claim 30, wherein the acid fungal alpha-amylase is derived from Aspergillus niger or Aspergillus oryzae.

32. The method of claim 15, wherein the acid alpha-amylase is an alpha-amylase having an amino acid sequence which has at least 80% identity to SEQ ID NO:1.

33. The method of claim 15, wherein the acid alpha-amylase is an alpha-amylase having an amino acid sequence which has at least 90% identity to SEQ ID NO:1.

34. The method of claim 15, wherein the acid alpha-amylase is an alpha-amylase having an amino acid sequence which has at least 95% identity to SEQ ID NO:1.

35. The method of claim 23, wherein the yeast is derived from a strain of Saccharomyces spp.


SEQUENCE LISTING (1 sequence)

SEQ ID NO: 1 - protein, 484 aa, Aspergillus niger
  misc_feature: SEQ ID NO1
LSAASWRTQSIYFLLTDRFGRTDNSTTATCNTGNEIYCGGSWQGIIDHLDYIEGMGFTAIWISPITEQLPQDTADGEAYHGYWQQKIYDVNSNFGTADNLKSLSDALHARGMYLMVDVVPDHMGYAGNGNDVDYSVFDPFDSSSYFHPYCLITDWDNLTMVEDCWEGDTIVSLPDLDTTETAVRTIWYDWVADLVSNYSVDGLRIDSVLEVQPDFFPGYNKASGVYCVGEIDNGNPASDCPYQKVLDGVLNYPIYWQLLYAFESSSGSISNLYNMIKSVASDCSDPTLLGNFIENHDNPRFAKYTSDYSQAKNVLSYIFLSDGIPIVYAGEEQHYAGGKVPYNREATWLSGYDTSAELYTWIATTNAIRKLAIAADSAYITYANDAFYTDSNTIAMAKGTSGSQVITVLSNKGSSGSSYTLTLSGSGYTSGTKLIEAYTCTSVTVDSSGDIPVPMASGLPRVLLPASVVDSSSLCGGSGRLYVE